(19) United States
(12) Patent Application Publication
(10) Pub. No.: US 2002/0096391 A1
(43) Pub. Date: Jul. 25, 2002

(54) Flexible ear insert and audio communication link

(76) Inventors: Richard C. Smith, Costa Mesa, CA (US); Diann Y. Smith, Costa Mesa, CA (US)

Correspondence Address: MORLAND C FISCHER, 2030 MAIN ST, SUITE 1050, IRVINE, CA, 92614

(21) Appl. No.: 09/767,808

(22) Filed: Jan. 24, 2001

(51) Int. Cl. A61B 7/02

(52) U.S. Cl. 181/135; 181/130

(57) ABSTRACT

A flexible ear insert to be comfortably and inconspicuously carried in the ear of a wearer, particularly law enforcement and security personnel. The flexible ear insert has a central air space surrounded by an arcuate band and a linear bridge. A canal tube having a sound channel running therethrough projects outwardly from the bridge of the ear insert to deliver audio messages from an audio source to the ear canal of the wearer. By virtue of the central air space, the arcuate band can be collapsed inwardly to cause the ear insert to conform to the shape of the wearer's ear and thereby create a reliable fit. The central air space also enables nearby sounds from the wearer's surroundings to be delivered to the wearer's ear drum at the same time that the audio messages are being delivered thereto from the audio source via the sound channel through the canal tube.

### BACKGROUND OF THE INVENTION

1. Field of the Invention

This invention relates to a flexible ear insert that is adapted to be comfortably and inconspicuously worn in the ear of a user so as to be reliably retained therein when the user is running or experiencing sharp head turns. The flexible ear insert has particular application as a communication link by which to supply clear audio messages from a remote transmitter (e.g., a radio) directly to the ear canal of the wearer.

2. Background Art

It is frequently necessary to transmit detailed audio messages to a recipient who is separated by a great distance from the source (e.g., a remote radio transmitter) of the messages. For example, law enforcement and security personnel working in the field are usually required to stay in constant contact with a central command station. Various ear pieces have heretofore been employed by which to deliver an audio message from the remote transmitter to the ear of the wearer. However, such ear pieces are often large and uncomfortable, especially when they must be worn for long periods of time. More particularly, many ear pieces in use today are manufactured in a single size from a relatively hard plastic and/or metallic material that extends into and over the top of the ear. Therefore, these ear pieces do not often fit well within the ear and are not easily adjustable to conform to the shape of the ear. Consequently, conventional ear pieces are known to fall out of the wearer's ear in the event he is running, turning his head, or subjected to impact forces while on the job. The undesirable loss of the ear piece could jeopardize a chase or a rescue effort, or impede the ability of the wearer to receive important information for undertaking a task.

What is more, the wearing of some ear pieces prevents the wearer from simultaneously receiving audio sounds from his environment. That is to say, it is not uncommon for an earpiece to substantially block the wearer's ear canal so that he is capable of hearing only those sounds being transmitted through the ear piece, but not nearby sounds which originate from his surroundings. In this case, the wearer may not be able to detect shouts for help or gun shots or other local messages at the same time that he is receiving a message from a remote transmitter.

Accordingly, what is needed is a comfortable ear piece that is adapted to easily conform to the shape of the wearer's ear to establish a reliable fit, whereby the ear piece will not easily fall out while the wearer is on the job and that will enable the wearer to simultaneously receive distant messages transmitted through his ear piece, as well as local sounds that originate from the wearer's environment.

### SUMMARY OF THE INVENTION

An ear insert is disclosed by which to deliver clear audio messages from a remote source (e.g., a radio transmitter) directly to the ear of the wearer. The ear insert is characterized by a flexible construction so as to conform to the shape of the ear of different wearers. By virtue of the foregoing, the ear insert will fit comfortably and inconspicuously within the wearer's ear, especially over long periods of time. Moreover, the ear insert will be reliably retained within the wearer's ear in order to resist falling out when the wearer is either running or making sharp head turns. Thus, the ear insert of this invention has particular application for use by law enforcement and security personnel.

The ear insert of this invention has a generally D-shape and includes an arcuate band and a linear bridge extending between opposite ends of the band. A relatively thick cushion is formed at the top of the insert at the intersection of the band and one end of the bridge. A relatively wide tab protrudes from the bottom of the insert at the intersection of the band and the opposite end of the bridge. At the center of the insert, surrounded by the band and the bridge, is an air space. A pad is formed at approximately the mid-point of the bridge, and a canal tube is molded thereto. A sound channel runs continuously through the pad and the canal tube of the ear insert. One end of a flexible, high frequency enhancing coiled tubing is detachably connected to the canal tube. The opposite end of the tubing is connected to an acoustic coupler. The acoustic coupler is adapted to be electrically connected to a local (e.g., radio) receiver carried by the wearer

During installation, the flexible ear piece is inserted such that the arcuate band is received around the conchaebowl of the ear. Because of its flexible nature, the arcuate band is adapted to be collapsed and reshaped within the central air space of the ear insert so as to conform to the shape of the ear. The cushion at the top of the insert will fit against the ridge of the helix of the ear, and the tab which protrudes from the bottom of the insert will fit within a notch that lies below the bowl of the ear. The canal tube is aligned with the bowl of the ear above the wearer's ear canal to establish a direct audio path from the receiver to the ear canal via the acoustic tubing and the sound channel that runs through the canal tube. As an additional advantage, local sounds that originate from the environment surrounding the wearer will be simultaneously transmitted to his ear canal through the air space at the center of the ear piece.

### BRIEF DESCRIPTION OF THE DRAWINGS

FIG. 1 shows a front view of the flexible ear insert which forms the present invention for receipt within the left ear of a user;

FIG. 2 shows a bottom view of the flexible ear insert of FIG. 1;

FIG. 3 shows a rear view of the flexible ear insert of FIG. 1;

FIG. 4 shows a side view of the flexible ear insert of FIG. 1,

FIG. 5 shows the ear insert of this invention adapted to be worn in the right ear of a wearer and being detachably connected to an acoustic coupler by way of flexible acoustic tubing;

FIG. 6 is an enlarged detail illustrating the detachable connection of the flexible acoustic tubing to the ear insert of FIG. 5; and

FIGS. 7 and 8 show the flexible ear insert of FIG. 5 being worn within the right ear of the wearer.

### DETAILED DESCRIPTION

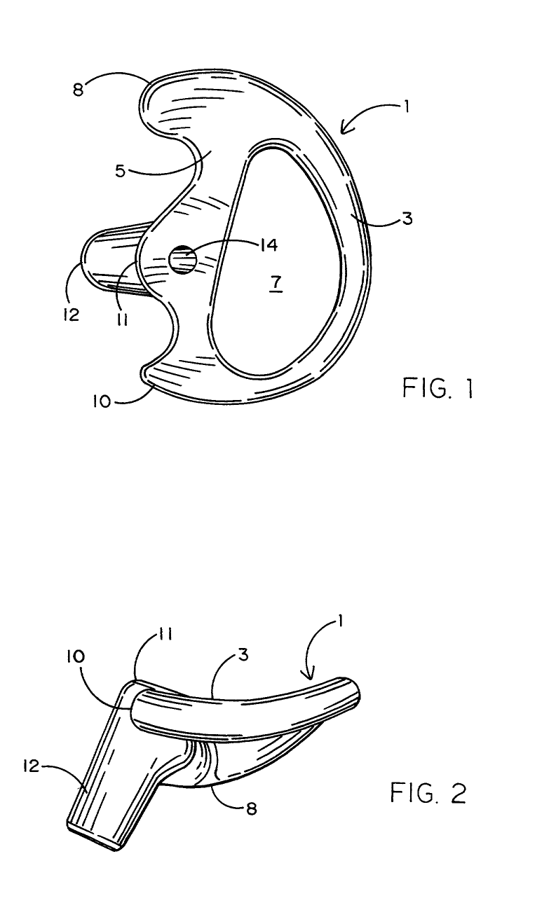

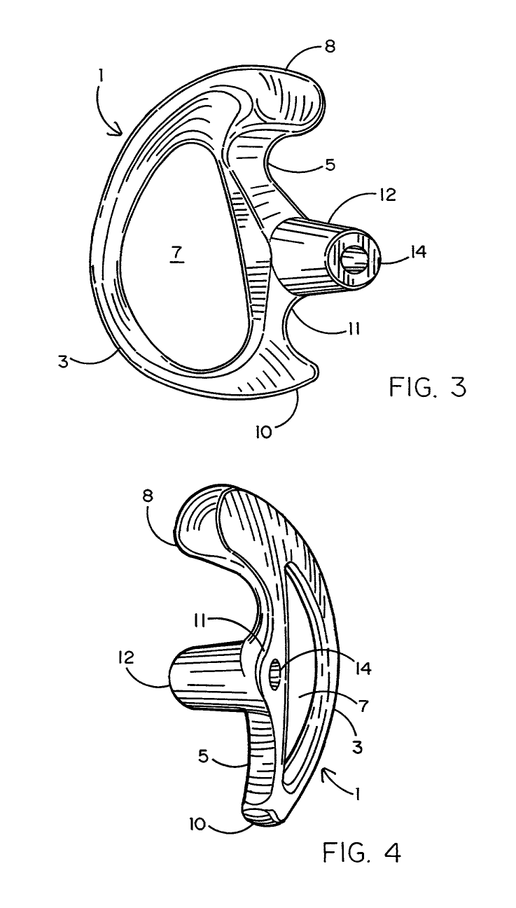

The flexible ear insert 1 which has particular application as a communication link to deliver clear audio messages to a user from a remote audio source and which forms the present invention is initially described while referring concurrently to FIGS. 1-4 of the drawings. In the preferred embodiment, the ear insert 1 is manufactured from a soft, pliable medical grade plastic material having a durometer of Shore A 35/45 according to a conventional injection molding process so as to possess a flexible characteristic that will not harden over time and thereby facilitate the ease of installation and maximize comfort when the ear insert 1 is worn in the user's ear for a long period of time. In addition, the ear insert 1 can be comfortably received in the ears of a majority of users regardless of the size and shape of their ears. The ear insert 1 can be molded so as to have different colors and sizes to correspond with the skin tone of the user and the size of the user's ear in which the ear piece will be carried. Changing the color of the plastic material from which the ear insert 1 is molded enhances the ability of the ear piece to remain substantially invisible within the wearer's ear. However, it should be recognized that the size, color and the flexible material from which the ear insert 1 is to be manufactured are not to be considered as limitations of this invention.

The flexible ear insert 1 has a generally D-shape so as to be adapted to fit snugly within the wearer's ear. More particularly, the ear insert 1 includes an arcuate band 3 and a generally linear bridge 5 which extends between opposite ends of the band 3. At the center of the ear insert 1, surrounded by the band 3 and the bridge 5, is an open air space 7. The air space 7 has three important functions. First, the air space 7 provides an area into which the flexible band 3 can be collapsed under pressure from the wearer's ear so as to tailor the insert to the particular ear of the wearer in order to improve the fit and maximize comfort. Secondly, the air space 7 provides an open area through which nearby sounds from the wearer's environment can pass directly to the wearer's ear canal. Thirdly, the air space 7 minimizes the weight of the ear insert 1 to maximize comfort in situations where the insert must be worn continuously for long periods of time.

A relatively thick cushion 8 is formed at the top of the ear insert 1 at the intersection of the band 3 and one end of the bridge 5. A short tab 10 protrudes outwardly from the bottom of the ear insert 1 at the intersection of the band 3 and the opposite end of the bridge 5. A relatively wide pad 11 is formed at approximately the mid-point of the bridge 5 between the cushion 8 and tab 10.

As an important feature of the flexible ear insert 1, a hollow canal tube 12 is molded to and projects outwardly from the pad 11 at the mid-point of the bridge 5 between the cushion 8 and tab 10. A sound channel 14 extends continuously through the canal tube 12 and the pad 11 of bridge 5. The canal tube 12 is of sufficient length so that, with the ear insert properly installed, the sound channel 14 running through canal tube 12 will extend into the bowl of the ear so as to be positioned above and axially aligned with the wearer's ear canal in order to establish a sound passage between an audio receiver and the wearer's ear canal. However, it is important to recognize that the canal tube 12 does not extend into and block the wearer's ear canal.

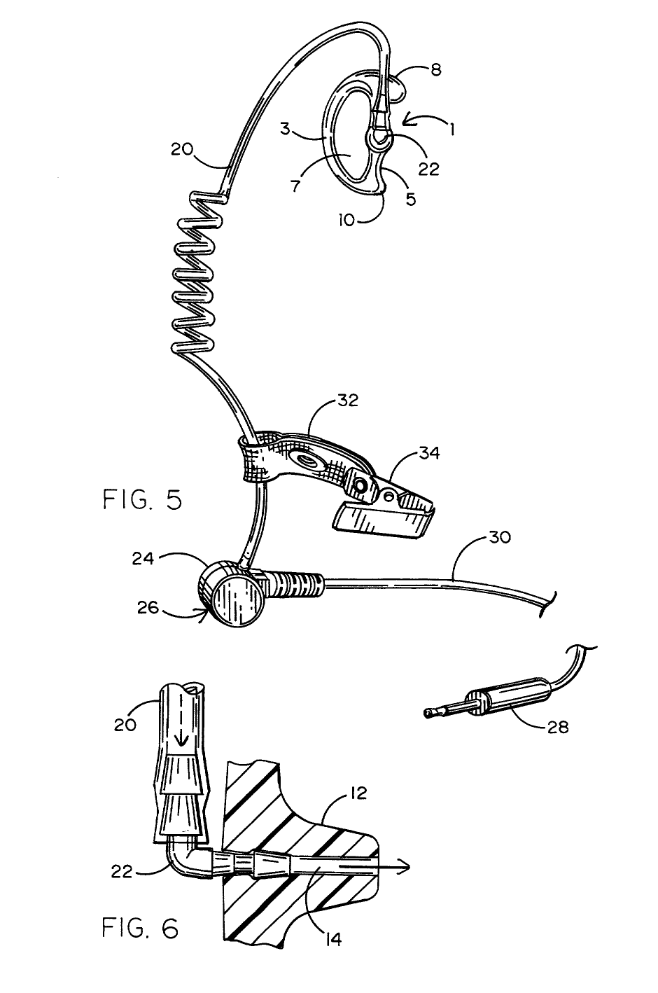

The installation and use of the flexible ear insert 1 of this invention are now disclosed while referring concurrently to FIGS. 5-8 of the drawings. In order for the wearer to receive messages from a remote source (e.g., a radio transmitter), the ear insert 1 is first connected to flexible, moisture resistant (coiled) acoustic tubing 20 that is capable of enhancing high frequency sound. As is best shown in FIG. 6, one end of a plastic elbow 22 is received by the acoustic tubing 20. The opposite end of the elbow 22 is detachably connected to the canal tube 12 of ear insert 1 so as to communicate with the sound channel 14 thereof The flexible nature of the ear insert advantageously allows elbows from different manufacturers to be attached to canal tube 12.

As is best shown in FIG. 5, the acoustic tubing 20 terminates at an electro-audio converter 24 of a commercially available acoustic coupler 26. The acoustic coupler 26 is connected to a conventional plug 28 by means of an electrical wire 30. The plug 28 is adapted to be connected to a radio receiver (not shown), or the like, that is carried on the body of the wearer in order to complete an audio communication path from the remote transmitter to the ear of the wearer in which the ear insert 1 will be carried. Such audio communication path includes wire 30, acoustic coupler 26, acoustic tubing 20, elbow 22, and the sound channel 14 which runs through the canal tube 12. A universal snap ring 32 and a standard shirt clip 34 may be attached to the acoustic tubing 20 to hold the tubing 20 and acoustic coupler 26 at a convenient location close to the body of the wearer.

With the aforementioned communication path in place between a local radio, or a similar receiver, and the sound channel 14 of canal tube 12, the flexible ear insert 1 is now ready to be installed within the ear of the wearer. However, it is first necessary to point out that different ear inserts are placed into the left and right ears of the wearer. The left and right inserts are identical in construction and function, but are mirror images of one another in appearance. In the case of FIGS. 1-4 of the drawings, the ear insert shown is designed to fit into the left ear of the wearer. For purposes of illustration, the ear insert shown in FIGS. 5-8 is designed to fit into the right ear of the wearer.

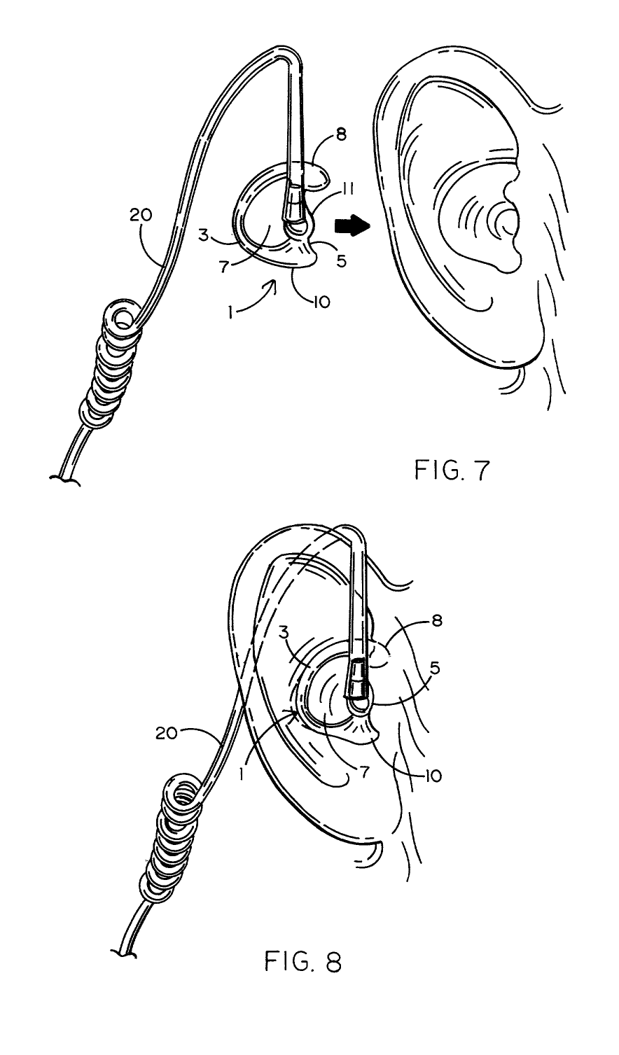

In this regard and as is best shown FIGS. 7 and 8, the flexible ear insert 1 is installed in and reliably retained at the right ear of the wearer Because of its unique shape and construction, the ear insert 1 is both comfortably and snugly positioned in the ear so as to remain both inconspicuous and reliably retained in place, even if the wearer is running or his head is subject to violent turning movements. Similarly, the wearer's ear is unlikely to sustain damage should the wearer be struck by a blow to his ear with insert 1 in place.

In the installed condition of FIG. 8, the arcuate band 3 of ear insert 1 is received around the conchaebowl of the ear to thereby prevent the insert from falling out of the ear. Because of its flexible nature, the arcuate band 3 is adapted to be compressed and reshaped within the central open air space 7 so as to conform to the shape of the wearer's ear. The cushion 8 formed at the top of ear insert 1 will be received against a ridge at the top of the ear, sometimes known as the helix. The tab 10 which protrudes from the bottom of the insert 1 is positioned to fit within a small notch that lies below the bowl of the ear.

With the ear insert 1 held snugly in place, the pad 11 at the mid-point of the bridge 5 through which sound channel 14 is formed fits behind the targus of the ear, whereby the canal tube 12 to which the acoustic tubing 20 is connected, will extend into the bowl of the ear to be positioned directly above and in axial alignment with the ear canal in order to advantageously provide loud and clear audio signals from the audio receiver directly to the ear of the wearer. What is more, the open air space 7 surrounded by the band 3 and bridge 5 at the center of ear insert 1 will be automatically positioned above and aligned with the bowl of the ear to establish a sound passage between the wearer's environment and his ear canal. Therefore, at the same time that the wearer receives communications transmitted from a remote source to the ear insert 1, he will also be able to hear nearby sounds that are transmitted from his surroundings to his ear canal via the air space 7, as well as the open area between the protrusion 10 and pad 11 along bridge 5. Such sounds may include gun shots, shouts for help, spoken words, etc.

The flexible ear insert 1 described above has particular application as a communication link for use by law enforcement and security personnel who must receive regular communications over long periods of time. The ear insert 1 allows the wearer to simultaneously receive audio messages transmitted from a remote source as well as nearby sounds generated from his surroundings. Because it is both comfortable and inconspicuous when worn, the flexible ear insert of this invention may also be used to receive communications from other audio sources, such as, but not limited to, cell phones, computers, and CD ROM players In this same regard, the ear insert 1 is also capable of having a miniature hearing aid vibration speaker connected to the canal tube 12 thereof to enhance the hearing of the wearer.

I claim:

1. An audio communication link to be coupled to the ear of a wearer by which audio messages from an audio source are delivered to the wearer, said audio communication link including an ear insert to be carried in the wearer's ear, said ear insert having an outer periphery to be received by an retained within the wearer's ear, a central air space surrounded by said outer periphery, and a canal tube depending outwardly from said outer periphery to be aligned with the ear canal of the wearer, said canal tube having a sound channel running therethrough by which the audio messages from the audio source are delivered to the wearer's ear canal.

2. The audio communication link recited in claim 1, wherein said ear insert is manufactured from a flexible material, such that the outer periphery thereof is collapsible inwardly of said central air space, whereby the shape of said ear insert can be reconfigured to correspond with the shape of the wearer's ear so as to provide a reliable and comfortable fit.

3. The audio communication link recited in claim 1, wherein said ear insert also has a relatively thick cushion formed on the outer periphery thereof to be received against the helix at the top of the wearer's ear.

4. The audio communication link recited in claim 3, wherein said ear insert also has a relatively wide tab protruding from the outer periphery thereof to be received in a notch that is formed below the bowl at the bottom of the wearer's ear.

5. The audio communication link recited in claim 4, wherein the central air space surrounded by the outer periphery of said ear insert is automatically positioned above the bowl of the wearer's ear when said thick cushion and said wide tab of said outer periphery are received in the wearer's ear, said central air space being of sufficient size to enable the wearer to simultaneously hear nearby sounds generated from his surroundings at the same time that the audio messages are being delivered from the audio source via the sound channel running through said canal tube.

6. The audio communication link recited in claim 1, also including acoustic tubing connected between an acoustic coupler and said canal tube at the outer periphery of said ear insert, said acoustic tubing supplying the audio messages from the audio source to the wearer's ear canal via the sound channel running through said canal tube.

7. The audio communication link recited in claim 6, also including a hollow elbow having first and opposite ends, the first end of said hollow elbow detachably connected to said acoustic tubing and the opposite end of said hollow elbow detachably connected to said canal tube.

8. The audio communication link recited in claim 1, wherein said outer periphery of said ear insert is D-shaped.

9. A flexible ear insert to be carried within the ear of a wearer by which to deliver audio messages from an audio source to the wearer's ear canal, said ear insert having an arcuate band and a linear bridge that are coextensively connected to one another so that said ear insert has a D-shape, a central air space surrounded by said arcuate band and said linear bridge to enable the wearer to hear nearby sounds generated from his surroundings and to permit said arcuate band to be collapsed inwardly of said central air space, and a canal tube projecting outwardly from said linear bridge to be received in axial alignment with the ear canal of the wearer, said canal tube having a sound channel running therethrough by which the audio messages from the audio source are delivered to the wearer's ear canal.

10. The ear insert recited in claim 9, also having a relatively thick cushion formed at the intersection of respective first ends of said arcuate band and said linear bridge to be received against the helix at the top of the wearer's ear and a relatively wide tab projecting from respective opposite ends of said arcuate band and said linear bridge to be received in a notch that is formed below the bowl at the bottom of the wearer's ear, the receipt of the thick cushion and the wide tab of said ear insert by the wearer's ear causing said central air space to be positioned above the bowl of the wearer's ear.
